(19) United States
(12) Patent Application Publication
(10) Pub. No.: US 2021/0302558 A1
(43) Pub. Date: Sep. 30, 2021

(54) VITAL-SIGN RADAR SENSOR USING A WIRELESS FREQUENCY-LOCKED LOOP

(71) Applicant: National Kaohsiung University of Science and Technology, Kaohsiung City (TW)

(72) Inventors: Kang-Chun Peng, Kaohsiung City (TW); Siang-En Chen, Miaoli County (TW); Fu-Kang Wang, Kaohsiung City (TW); Tzyy-Sheng Horng, Kaohsiung City (TW)

(21) Appl. No.: 16/885,607

(22) Filed: May 28, 2020

(30) Foreign Application Priority Data
Mar. 30, 2020 (TW) 109110785

(51) Int. Cl. G01S 13/34; A61B 5/05

(52) U.S. Cl. CPC G01S 13/34; A61B 5/05

(57) ABSTRACT

A vital-sign radar sensor using wireless frequency-locked loop includes a voltage-controlled oscillator (VCO), an antenna component, a mixer, a loop filter and a frequency demodulation component. The VCO outputs an oscillation signal to the antenna component via a output port, the antenna component transmits the oscillation signal to a subject as a transmitted signal and receives a reflected signal from the subject as a received signal, the mixer receives and mix the oscillation signal and the received signal into a mixed signal, the loop filter receives and filter the mixed signal to output a filtered signal, the filtered signal is delivered to the VCO via a tuning port, the frequency demodulation component receives and demodulates the oscillation signal to output a vital-sign signal.

### FIELD OF THE INVENTION

This invention generally relates to a vital-sign radar sensor, and more particularly to a vital-sign radar sensor using a wireless frequency-locked loop.

### BACKGROUND OF THE INVENTION

Conventional continuous wave (CW) radar can transmit a transmission signal to a moving subject and receive a reflected signal from the moving subject, the movement of the subject may generate Doppler Effect on the transmission signal to allow the reflected signal to contain Doppler phase shifts. For this reason, the information of the movement of the subject can be extracted from the reflected signal received by CW radar. An oscillation signal output from an oscillator of CW radar is transmitted as the transmission signal and also used as a local oscillation signal for frequency down-conversion or signal demodulation, so the frequency stability of CW radar is important. The oscillator may have phase noise due to internal components or external injection signals, for example, thermal noise, shot noise or flicker noise from internal components of the oscillator (resistor, capacitor, inductor and transistor) may vary amplitude, phase or frequency of the oscillation signal. The phase noise may cover Doppler phase shifts caused by tiny movement of the subject, such as vital sign, to result in a detection error.

Self-injection-locked (SIL) radar has a good sensitivity to tiny vibration for vital-sign detection because the reflected signal from the moving subject can be injected into injection port of the oscillator of the SIL radar to vary the frequency of the oscillator. However, null point may exist if the distance from the subject to the transmit antenna of SIL radar is an integer multiple of a half-wavelength.

### SUMMARY

The present invention provides a wireless frequency-locked loop composed of voltage-controlled oscillator, antenna component, propagation delay between antenna component and subject, mixer and loop filter to allow Doppler phase shifts caused by subject's movement to modulate the voltage-controlled oscillator such that frequency shifts can present vital sign of subject. Additionally, the wireless frequency-locked loop also can reduce phase noise of the voltage-controlled oscillator to enhance sensitivity of vital-sign detection.

A vital-sign radar sensor of the present invention includes a voltage-controlled oscillator (VCO), an antenna component, a mixer, a loop filter and a frequency demodulation component. The VCO includes an output port and a tuning port and is configured to output an oscillation signal via the output port. The antenna component is coupled to the VCO and configured to receive and transmit the oscillation signal as a transmitted signal to a subject and configured to receive a reflected signal from the subject as a received signal. The mixer is coupled to the VCO and the antenna component and configured to receive and mix the oscillation signal and the received signal to output a mixed signal. The loop filter is coupled to the mixer and configured to receive and filter the mixed signal to output a filtered signal, the filtered signal is configured to be delivered to the VCO via the tuning port. The frequency demodulation component is coupled to the VCO and configured to receive and demodulate the oscillation signal to output a vital-sign signal.

A wireless frequency-locked loop composed of the VCO, the antenna component, the propagation delay between the transmit antenna and the subject, the propagation delay between the subject and the receive antenna, the mixer and the loop filter is provided to detect vital sign of the subject in the present invention. The using of the wireless frequency-locked loop can eliminate null point and reduce phase noise to improve signal-to-noise ratio of the vital-sign signal and detection range of the vital-sign radar sensor.

### DESCRIPTION OF THE DRAWINGS

FIG. 1 is a circuit diagram illustrating a vital-sign radar sensor in accordance with a first embodiment of the present invention.

FIG. 2 is a circuit diagram illustrating a vital-sign radar sensor in accordance with a second embodiment of the present invention.

FIG. 3 is a circuit diagram illustrating a vital-sign radar sensor in accordance with a third embodiment of the present invention.

FIG. 4 is a circuit diagram illustrating a vital-sign radar sensor in accordance with a fourth embodiment of the present invention.

### DETAILED DESCRIPTION OF THE INVENTION

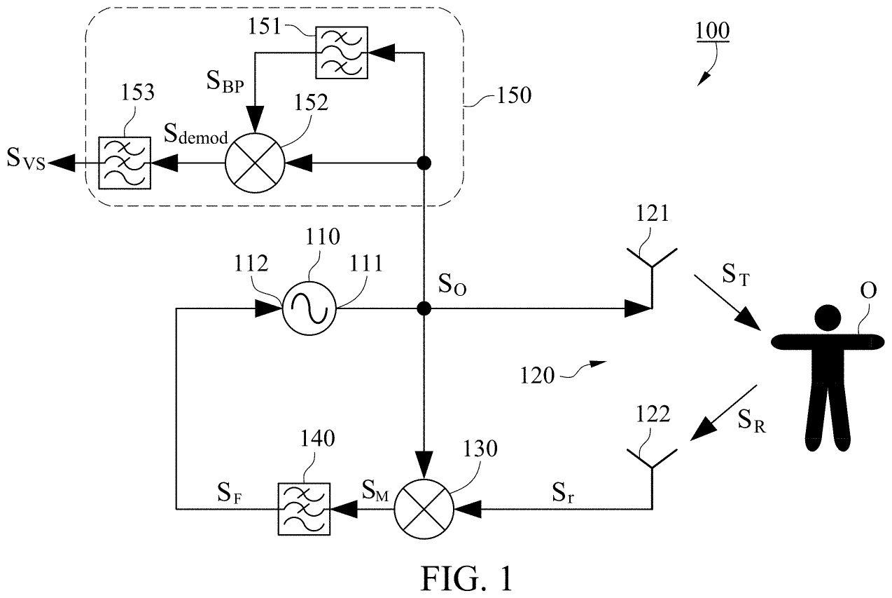

With reference to FIG. 1, a vital-sign radar sensor 100 in accordance with a first embodiment of the present invention includes a voltage-control oscillator (VCO) 110, an antenna component 120, a mixer 130, a loop filter 140 and a frequency demodulation component 150.

The VCO 110 includes an output port 111 and a tuning port 112 and is configured to output an oscillation signal S_{O} from the output port 111. The antenna component 120 includes a transmit antenna 121 and a receive antenna 122, the transmit antenna 121 is coupled to the output port 111 of the VCO 110 and configured to receive and transmit the oscillation signal S_{O} as a transmitted signal S_{T} to a subject O. While the subject O has a motion relative to the transmit antenna 121, the motion results in a Doppler Effect on the transmitted signal S_{T} to allow a reflected signal S_{R} from the subject O to contain Doppler phase shifts. The receive antenna 122 is configured to receive the reflected signal S_{R} as a received signal Sᵣ from the subject O, the received signal Sᵣ also contains the Doppler phase shifts caused by the motion of the subject O.

The mixer 130 is coupled to the VCO 110 and the antenna component 120 for receiving the oscillation signal S_{O} and the received signal Sᵣ and configured to mix the two signals into a mixed signal S_{M}. The mixed signal S_{M} represents the phase variation from the oscillation signal S_{O} to the received signal Sᵣ so it contains the information of the motion of the subject O. In the first embodiment, the combination of the signal propagation from the transmit antenna 121 to the subject O and from the subject O to the receive antenna 122 and the down-conversion process of the mixer 130 constructs a frequency discriminator configured to demodulate the Doppler phase shifts caused by the motion of the subject O and the phase noise of the VCO 110.

The loop filter 140 is coupled to the mixer 130 and configured to receive and filter the mixed signal S_{M} to output a filtered signal S_{F}. In the first embodiment, the loop filter 140 is a low-pass filter that is configured to filter high-frequency content of the mixed signal S_{M} so as to extract the low-frequency content from vital signs. The filtered signal S_{F} delivered to the tuning port 112 of the VCO 110 from the loop filter 140 is configured to modulate the oscillation signal S_{O} of the VCO 110 to generate frequency shifts on the oscillation signal S_{O}. A wireless frequency-locked loop (FLL) is constructed of the VCO 110, the antenna component 120, the propagation delay between the transmit antenna 121 and the subject O, the propagation delay between the subject O and the receive antenna 122, the mixer 130 and the loop filter 140. The delay of delay element of the wireless FLL is positively correlated with the inhibition of the phase noise such that the wireless FLL of the first embodiment, using the propagation delay between the transmit antenna 121 and the subject O and between the subject O and the receive antenna 122 as the delay element, reveals better inhibition of the phase noise when the subject O is located at a longer distance from the antenna component 120.

In the first embodiment, the feedback of the demodulated signal having the Doppler phase shifts caused by the motion of the subject O re-modulates the VCO 110 such that the VCO 110 can trace the Doppler phase shifts to enhance the strength of vital sign sensing due to its high tuning sensitivity, and the vital-sign detection range is not restricted because there is no null point. In addition, the re-modulation of the VCO 110 by using the demodulated signal having the Doppler phase shifts also reduces the phase noise of the VCO 110 to increase signal-to-noise ratio (SNR) of the vital-sign radar sensor 100.

With reference to FIG. 1 again, the frequency demodulation component 150 is coupled to the VCO 110 for receiving the oscillation signal S_{O} and configured to demodulate the oscillation signal S_{O} to output a vital-sign signal S_{VS}. In the first embodiment, the frequency demodulation component 150 includes a surface acoustic wave (SAW) filter 151, a demodulation mixer 152 and a low-pass filter 153. The SAW filter 151 is coupled to the VCO 110 and configured to receive the oscillation signal S_{O} and output a band-pass filtered signal S_{BP}. The demodulation mixer 152 is coupled to the VCO 110 and the SAW filter 151 in order to receive the oscillation signal S_{O} and the band-pass filtered signal S_{BP}, and configured to mix the two signals to output a demodulated signal S_{demod}. The low-pass filter 153 is electrically connected to the demodulation mixer 152 to receive the demodulated signal S_{demod}, and configured to filter the high-frequency content of the demodulated signal S_{demod} so as to output the vital-sign signal S_{VS}.

The wireless FLL of the first embodiment can reduce the phase noise of the VCO 110, as a result, vital-sign sensitivity and detection range of the vital-sign radar sensor 100 are increased for long-distance detection of vital sign of the subject O.

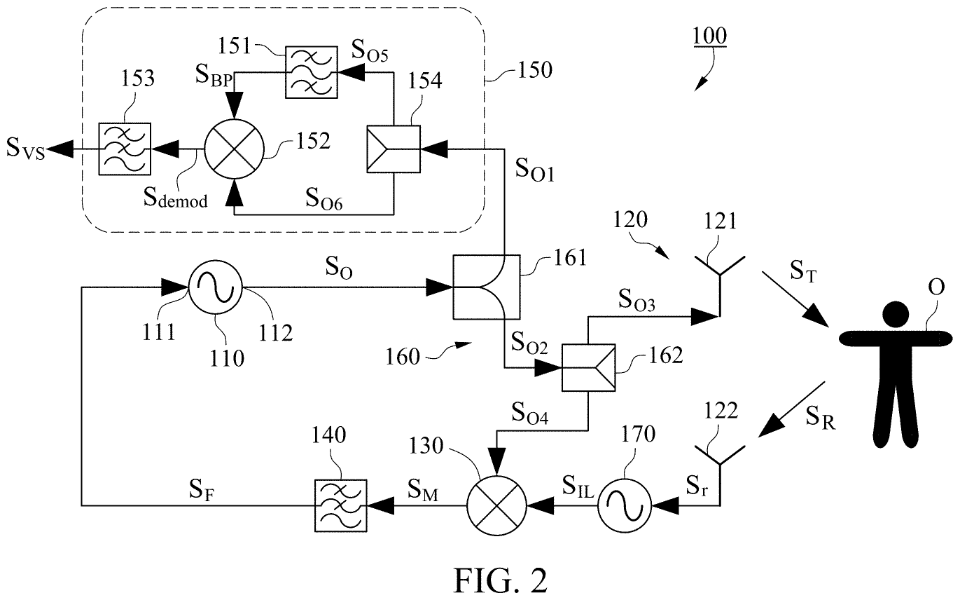

FIG. 2 presents a second embodiment of the present invention. Different to the first embodiment, the vital-sign radar sensor 100 of the second embodiment further includes a power-split component 160 and an injection-locked oscillator (ILO) 170. The power-split component 160 is electrically connected to the VCO 110 and configured to receive and divide the oscillation signal S_{O} into three paths, the oscillation signal S_{O} in three paths are delivered to the antenna component 120, the mixer 130 and the frequency demodulation component 150, respectively. In the second embodiment, the power-split component 160 includes a first power splitter 161 and a second power splitter 162. The first power splitter 161 is electrically connected to the VCO 110 and configured to receive and divide the oscillation signal S_{O} into two paths, the oscillation signal S_{O1} of one path is delivered to the frequency demodulation component 150 and the oscillation signal S_{O2} of the other path is delivered to the second power splitter 162. The second power splitter 162 is electrically connected to the first power splitter 161 and configured to split the oscillation signal S_{O2} received from the first power splitter 161 into two paths, the oscillation signal S_{O3} of one path is delivered to the antenna component 120 and transmitted by the transmit antenna 121 as the transmitted signal S_{T}, and the oscillation signal S_{O4} of the other path is delivered to the mixer 130 for mixing.

The ILO 170 is electrically connected to the receive antenna 122 to receive the received signal Sᵣ and injection-locked by the received signal Sᵣ to output an injection-locked signal S_{IL}. The injection-locked signal S_{IL} is delivered to the mixer 130 to mix with the oscillation signal S_{O4}. The ILO 170 can amplify the Doppler phase shifts of the received signal Sᵣ result from the motion of the subject O to effectively enhance the SNR of the vital-sign signal S_{VS} detected by the vital-sign radar sensor 100.

With reference to FIG. 2 again, the frequency demodulation component 150 further includes a power splitter 154 in the second embodiment. The power splitter 154 is coupled to the first power splitter 161 and configured to receive and divide the oscillation signal S_{O1} into two paths, the oscillation signal S_{O5} in one path is delivered to the SAW filter 151, and the oscillation signal S_{O6} in the other path is delivered to the demodulation mixer 152. In a similar way, the phase noise of the VCO 110 is also reduced by the wireless FLL to improve the vital-sign sensitivity of the vital-sign radar sensor 100.

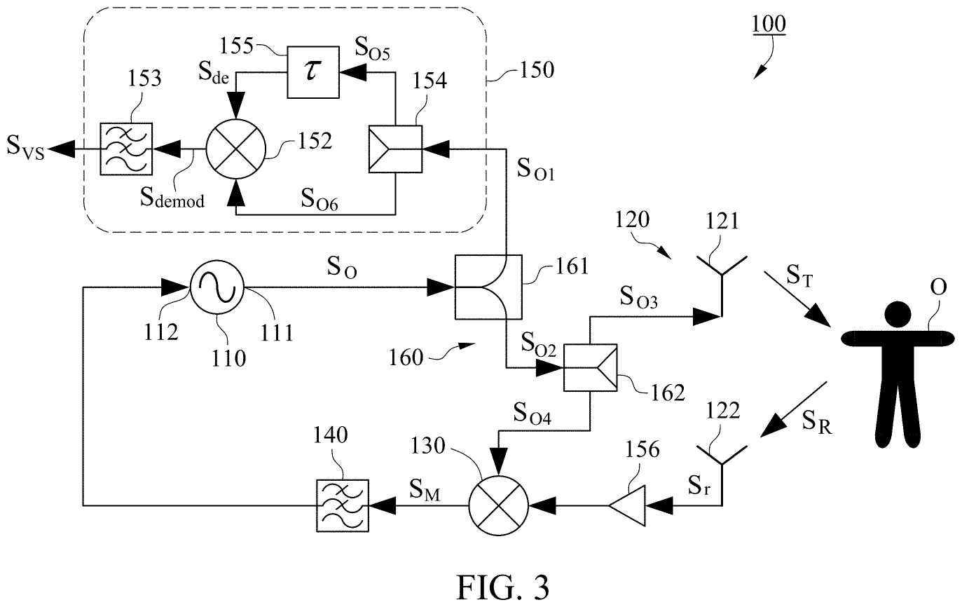

In a third embodiment of the present invention as shown in FIG. 3, the SAW filter 151 of the frequency demodulation component 150 is replaced by a delay line 155 and the ILO 170 is replaced by a low noise amplifier (LNA) 180. The delay line 155 is coupled to the VCO 110 via the power splitter 154 and the first power splitter 161 for receiving the oscillation signal S_{O5} and configured to output a delayed signal S_{de}. The demodulation mixer 152 is coupled to the power splitter 154 and the delay line 155 so as to receive the oscillation signal S_{O6} and the delayed signal S_{de}, and configured to mix the oscillation signal S_{O6} and the delayed signal S_{de} to output the demodulated signal S_{demod}. In the third embodiment, the frequency demodulation component 150 having the delay line 155 used to replace the SAW filter 151 of the second embodiment is also able to frequency demodulate the oscillation signal S_{O1}, and the LNA 180 used to replace the ILO 170 of the second embodiment is also able to amplify the Doppler phase shifts of the received signal Sᵣ caused by the motion of the subject O to further increase the SNR of the vital-sign radar sensor 100. In other embodiments, only the SAW filter 151 of the second embodiment is replaced by the delay line 155 or only the ILO 170 of the second embodiment is replaced by the LNA 180.

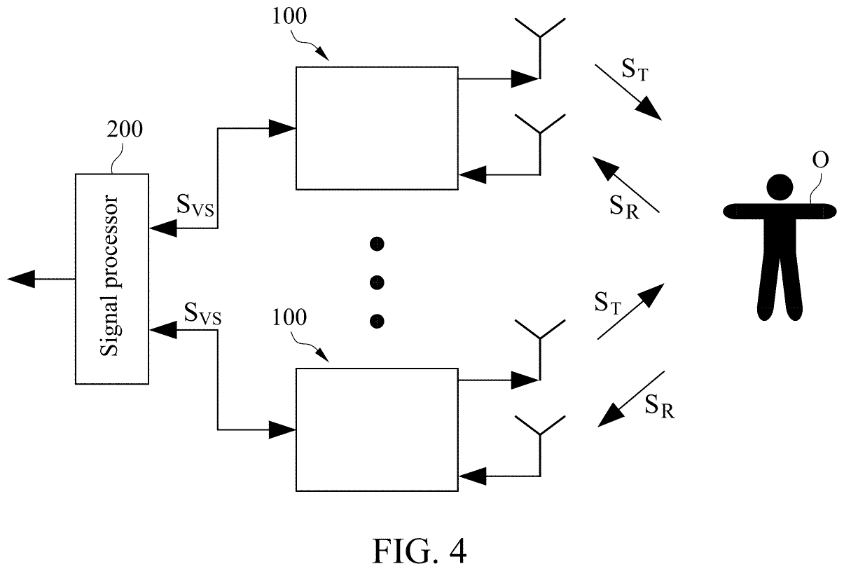

FIG. 4 shows a fourth embodiment of the present invention, multiple vital-sign radar sensors 100 are provided to detect vital sign(s) of one subject O or multiple subjects O. A signal processor 200 is electrically connected to the vital-sign radar sensors 100 and configured to control the phase difference between the transmitted signals S_{T} output from the vital-sign radar sensors 100 so as to form a beam which is available with angle adjustment to detect the subjects O located at different orientations. The signal processor 200 is also configured to receive the vital-sign signals S_{VS} from the vital-sign radar sensors 100, the vital-sign signals S_{VS} detected by the vital-sign radar sensors 100 present the vital sign of the subject O when the beam composed of the transmitted signals S_{T} is directed toward the subject O, consequently, the orientation of the subject O can be determined.

The present invention utilizes the wireless FLL composed of the VCO 110, the antenna component 120, the propagation delay between the transmit antenna 121 and the subject O, the propagation delay between the subject O and the receive antenna 122, the mixer 130 and the loop filter 140 to detect the vital sign of the subject O. The using of the wireless FLL can eliminate null point and reduce phase noise to enhance the SNR of the vital-sign signal S_{VS} and increase detection range of the vital-sign radar sensor 100.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A vital-sign radar sensor, comprising:
a voltage-controlled oscillator (VCO) including an output port and a tuning port and configured to output an oscillation signal via the output port;
an antenna component coupled to the VCO and configured to receive and transmit the oscillation signal to a subject as a transmitted signal and configured to receive a reflected signal from the subject as a received signal;
a mixer coupled to the VCO and the antenna component and configured to receive and mix the oscillation signal and the received signal to output a mixed signal;
a loop filter coupled to the mixer and configured to receive and filter the mixed signal to output a filtered signal, the filtered signal is configured to be delivered to the VCO via the tuning port; and
a frequency demodulation component coupled to the VCO and configured to receive and demodulate the oscillation signal to output a vital-sign signal.

2. The vital-sign radar sensor in accordance with claim 1 further comprising a power-split component, wherein the power-split component is electrically connected to the VCO and configured to receive and divide the oscillation signal into three paths, the oscillation signal of the three paths is configured to be delivered to the antenna component, the mixer and the frequency demodulation component, respectively.

3. The vital-sign radar sensor in accordance with claim 2, wherein the power-split component includes a first power splitter electrically connected to the VCO and a second power splitter electrically connected to the first power splitter, the first power splitter is configured to receive and divide the oscillation signal into two paths, the oscillation signal of one path is configured to be delivered to the frequency demodulation component and the oscillation signal of the other path is configured to be delivered to the second power splitter, the second power splitter is configured to divide the oscillation signal received from the first power splitter into two paths, the oscillation signal of one path is configured to be delivered to the antenna component and the oscillation signal of the other path is configured to be delivered to the mixer.

4. The vital-sign radar sensor in accordance with claim 1, wherein the antenna component includes a transmit antenna and a receive antenna, the transmit antenna is coupled to the VCO and configured to receive and transmit the oscillation signal as the transmitted signal, the receive antenna is configured to receive the reflected signal from the subject as the received signal.

5. The vital-sign radar sensor in accordance with claim 4 further comprising an injection-locked oscillator, wherein the injection-locked oscillator is electrically connected to the receive antenna and configured to receive and be injection-locked by the received signal to output an injection-locked signal, the injection-locked signal is configured to be delivered to the mixer.

6. The vital-sign radar sensor in accordance with claim 1, wherein the loop filter is a low-pass filter configured to filter a high-frequency content of the mixed signal.

7. The vital-sign radar sensor in accordance with claim 1, wherein the frequency demodulation component includes a surface acoustic wave (SAW) filter, a demodulation mixer and a low-pass filter, the SAW filter is coupled to the VCO and configured to receive the oscillation signal and output a band-pass filtered signal, the demodulation mixer is coupled to the VCO and the SAW filter and configured to receive and mix the oscillation signal and the band-pass filtered signal to output a demodulated signal, the low-pass filter is electrically connected to the demodulation mixer and configured to receive the demodulated signal and filter a high-frequency content of the demodulated signal to output the vital-sign signal.

8. The vital-sign radar sensor in accordance with claim 7, wherein the frequency demodulation component further includes a power splitter, the power splitter is coupled to the VCO and configured to receive and divide the oscillation signal into two paths, the oscillation signal of one path is configured to be delivered to the SAW filter and the oscillation signal of the other path is configured to be delivered to the demodulation mixer.

9. The vital-sign radar sensor in accordance with claim 1, wherein the frequency demodulation component includes a delay line, a demodulation mixer and a low-pass filter, the delay line is coupled to the VCO and configured to receive the oscillation signal and output a delayed signal, the demodulation mixer is coupled to the VCO and the delay line and configured to receive and mix the oscillation signal and the delayed signal to output a demodulated signal, the low-pass filter is electrically connected to the demodulation mixer and configured to receive the demodulated signal and filter a high-frequency content of the demodulated signal to output the vital-sign signal.

10. The vital-sign radar sensor in accordance with claim 9, wherein the frequency demodulation component further includes a power splitter, the power splitter is coupled to the VCO and configured to receive and divide the oscillation signal into two paths, the oscillation signal of one path is configured to be delivered to the delay line and the oscillation signal of the other path is configured to be delivered to the demodulation mixer.
